(19) United States
(12) Patent Application Publication
(10) Pub. No.: US 2016/0030312 A1
(43) Pub. Date: Feb. 4, 2016

(54) CARBONATED HAIR COSMETIC COMPOSITIONS

(71) Applicant: L'OREAL, Paris (FR)

(72) Inventors: Michell CHEN, Tokyo (JP); Gloria Frimpong ALLORBI, Parsippany, NJ (US); Anand MAHADESHWAR, Scotch Plains, NJ (US)

(21) Appl. No.: 14/447,946

(22) Filed: Jul. 31, 2014

(51) Int. Cl. A61K 8/362; A61Q 5/06; A61Q 5/00; A61K 8/19

(52) U.S. Cl. CPC A61K 8/362; A61K 8/19; A61Q 5/06; A61Q 5/00; A61K 2800/59

(57) ABSTRACT

Disclosed are methods for improving the improving the manageability of keratin fibers such as hair involving providing compositions comprising at least one bicarbonate compound, at least one acid, and at least one cosmetically acceptable carrier, wherein the weight ratio of the at least one bicarbonate compound to the at least one acid is greater than or equal to about 1:1.

### TECHNICAL FIELD

The present disclosure relates to methods for improving the manageability of keratin substrates, for instance human keratin fibers such as the hair, comprising providing a composition comprising at least one bicarbonate, at least one acid, and at least cosmetically acceptable carrier.

### BACKGROUND OF THE INVENTION

Consumers of cosmetic products actively seek out multi-functional, new products which are pleasing to the senses, both on application and in use, and which have innovative, interesting and/or pleasing textures, preferably without any sacrifice to functional performance. For example, for hair care and hair treatment products, one important functional element of such compositions is their ability to condition and style the hair without weighing it down, as well maintain the style of the hair, improve hair manageability and/or control the frizziness of hair. Under high humidity conditions, hair tends to absorb moisture causing it to be less manageable, which makes it more difficult to shape and style hair. Frizzy hair is particularly prone to problems when exposed to higher humidity. Applying a coating, such as a moisture barrier or a film on the hair is known to help to keep moisture out of the hair allowing for more efficient hair shaping and maintenance of hair shape, even in extreme humidity conditions.

Thus, the ability to shape and/or maintain the shape of hair, improve the manageability of hair, control the frizziness of hair, achieve a styling hold, maintain curls, and impart good texture on hair, while providing a clean, natural and light-weight feel to the hair remain as additional areas for improving the performance of hair cosmetic compositions.

Surprisingly and unexpectedly, the applicants have discovered that combining a carbonated system comprising a bicarbonate compound and an acid yielded a composition that imparts shaping or styling hold as well as resistance to moisture or humidity, anti-frizz, curl definition, and curl retention properties to keratin fibers such as hair. The current invention allows for easy and improved manageability and shaping of the fibers into a desired configuration. Moreover, the composition of the present invention is easy to remove from the keratin fibers by shampooing or rinsing with water or with a cosmetically acceptable remover. The compositions of the current invention can also provide good cosmetic properties to the keratin fibers such as a clean and natural feel, no tacky feel, no flaking, and touchable good hold.

### BRIEF SUMMARY OF THE INVENTION

The present invention relates to a method for improving the manageability of hair, the method comprising:

1) providing a cosmetic composition, containing:

a) at least one bicarbonate compound;

b) at least one acid; and

c) at least one cosmetically acceptable carrier;

wherein the weight ratio of the at least one bicarbonate compound (a) to the at least one acid (b) ranges from greater than or equal to about 1:1; and
2) providing instructions for applying the composition to hair.

The present invention also relates to methods for styling or shaping hair, improving curl retention and controlling or reducing the frizziness of hair involving applying onto hair the above-described composition.

### DETAILED DESCRIPTION OF THE INVENTION

It has been unexpectedly and surprisingly discovered that the methods of the present invention provides manageability, styling or shaping hold, curl definition, curl retention and anti-frizz control benefits onto keratin fibers such as hair.

Without being bound by theory, it is believed that combinations of a bicarbonate compound and an acid such as citric acid or lactic acid at certain weight ratios to each other provide compositions that impart films or coatings on the surface of keratin fibers such as hair.

The composition of the present invention is particularly useful as a hair gel composition.

The composition of the present invention is also particularly useful as an aerosolized hair cosmetic composition such as a hair mousse composition or hair spray composition when the composition contains at least one propellant.

As used herein, the expression “at least one” means one or more and thus includes individual components as well as mixtures/combinations.

It is also to be understood that, as used herein the terms “the,” “a,” or “an,” mean “at least one,” are understood to encompass the plural as well as the singular and should not be limited to “only one” unless explicitly indicated to the contrary. Thus, for example, the use of “an acid” is intended to mean at least one acid.

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8% - 2.2%).

The term “comprising” (and its grammatical variations) as used herein is used in the inclusive sense of “having” or “including” and not in the exclusive sense of “consisting only of”.

“Keratin fiber” as used herein, includes, but is not limited to hair, such as hair on the human head and eyelashes.

As used herein, the term “carbonated” or “carbonation” is understood to mean the combination of a bicarbonate compound and at least one acid.

“Film former” or “film forming polymer” as used herein means a polymer or resin that leaves a film on the substrate to which it is applied, for example, after a solvent accompanying the film former has evaporated, absorbed into and/or dissipated on the substrate. These terms may also refer to a polymer capable, by itself or in the presence of an auxiliary film forming polymer, of forming a continuous or a discontinuous film that adheres to a support and especially to keratin substrates such as keratin fibers or hair.

“Film former” or “film forming polymer” as used herein may also be referred to as fixing polymers when such polymers are employed to fix or keep keratin fibers in a particular configuration or shape or arrangement.

Among the film forming polymers that may be used in the cosmetic composition as disclosed herein, non-limiting mention may be made of cationic polymers, anionic polymers, nonionic polymers, amphoteric polymers, silicone polymers, or mixtures thereof.

The term “aqueous composition” or variations thereof means that the composition comprises water and optionally, substances of a formulation which, due to their hydrophilic character, can be mixed and/or dissolved and/or dispersed in water.

“Volatile”, as used herein, means having a flash point of less than about 100° C.

“Non-volatile”, as used herein, means having a flash point of greater than about 100° C.

“Substituted” as used herein, means comprising at least one substituent. Non-limiting examples of substituents include atoms, such as oxygen atoms and nitrogen atoms, as well as functional groups, such as acyloxyalky groups, carboxylic acid groups, amine or amino groups, acylamino groups, amide groups, halogen containing groups, ester groups, thiol groups, sulphonate groups, thiosulphate groups, siloxane groups, and polysiloxane groups. The substituent(s) may be further substituted.

The terms “organic compound” and “having an organic structure” mean compounds containing carbon atoms and hydrogen atoms and optionally heteroatoms such as S, O, N or P, alone or in combination.

As used herein, the terms “applying a composition onto keratin fibers” and “applying a composition onto hair” and variations of these phrases are intended to mean contacting the fibers or hair, with at least one of the compositions of the invention, in any manner.

As used herein, “formed from,” means obtained from chemical reaction of, wherein “chemical reaction,” includes spontaneous chemical reactions and induced chemical reactions. As used herein, the phrase “formed from,” is open ended and does not limit the components of the composition to those listed.

The term “stable” as used herein means that the composition does not exhibit phase separation and/or crystallization.

The term “treat” (and its grammatical variations) as used herein refers to the application of the compositions of the present invention onto keratin fibers such as hair.

The term “shaping” (and its grammatical variations) as used herein includes styling or placing a keratin fiber such as hair, in a particular arrangement, form or configuration; or altering the curvature of a keratin fiber or other substrate; or re-positioning a keratin fiber or other substrate to a different arrangement, form or configuration.

As used herein, the terms “method of shaping keratin fibers” or “method of shaping hair” is understood to mean any method for modifying the appearance of the keratin fibers or the hair with respect to their spatial arrangement or configuration or curvature or form. When the keratin fibers comprise hair on the human head, the term “method of shaping keratin fibers” or “method of shaping hair” is also understood to mean any method for curling or waving or embossing the hair or smoothing or straightening the hair, or spiking the hair.

As used herein, the term “improving the manageability of hair” or variations thereof includes, but is not limited to, controlling or reducing the frizziness/volume of hair and/or adding to the curl definition and/or retaining the curl of the hair (curl retention).

The compositions and methods of the present invention can comprise, consist of, or consist essentially of the essential elements and limitations of the invention described herein, as well as any additional or optional ingredients, components, or limitations described herein or otherwise useful.

Unless otherwise specified herein, all percentages and ratios of components are by weight relative to the total weight of the final composition.

In an embodiment, the present invention relates to a method for improving the manageability of hair, the method comprising:

1) providing a cosmetic composition, containing:

a) at least one bicarbonate compound;

b) at least one acid; and

c) at least one cosmetically acceptable carrier;

wherein the weight ratio of the at least one bicarbonate compound (a) to the at least one acid (b) ranges from greater than about 1:1 to about 3:1; and
2) providing instructions for applying the composition to hair.

In an embodiment, the present invention relates to a method for imparting curl retention properties to hair, the method comprising:

1) providing a cosmetic composition, containing:

a) at least one bicarbonate compound;

b) at least one acid; and

c) at least one cosmetically acceptable carrier;

wherein the weight ratio of the at least one bicarbonate compound (a) to the at least one acid (b) ranges from greater than about 1:1 to about 3:1; and
2) providing instructions for applying the composition to hair.

In an embodiment, the present invention relates to a method for controlling the frizziness of hair, the method comprising:

1) providing a cosmetic composition, containing:

a) at least one bicarbonate compound;

b) at least one acid; and

c) at least one cosmetically acceptable carrier;

wherein the weight ratio of the at least one bicarbonate compound (a) to the at least one acid (b) ranges from greater than about 1:1 to about 3:1; and
2) providing instructions for applying the composition to hair.

In one embodiment, the present invention relates to a method for retaining the curl and/or improving curl definition of hair, the method comprising:

1) providing a cosmetic composition, containing:

a) at least one bicarbonate compound;

b) at least one acid; and

c) at least one cosmetically acceptable carrier;

wherein the weight ratio of the at least one bicarbonate compound (a) to the at least one acid (b) ranges from greater than about 1:1 to about 3:1; and
2) providing instructions for applying the composition to hair

In certain embodiments, the compositions in the above described methods further comprise at least one auxiliary ingredient selected from at least one propellant, at least one emulsifier, at least one rheology modifier, at least one film forming polymer, and mixtures thereof.

In other embodiments, the compositions in the above described methods are in the form of aqueous hair cosmetic compositions such as gels or creams.

In other embodiments, the compositions in the above described methods are in the form of hair mousse cosmetic compositions.

In an embodiment, the at least one acid in the compositions of the methods of the present invention is selected from citric acid, lactic acid, and mixtures thereof.

In an embodiment, the at least one bicarbonate compound in the compositions of the methods of the present invention is sodium bicarbonate.

A method for cosmetically treating hair, the method comprising:

1) providing a cosmetic composition, containing:

a) at least one bicarbonate compound;

b) at least one acid; and

c) at least one cosmetically acceptable carrier;

wherein the weight ratio of the at least one bicarbonate compound (a) to the at least one acid (b) ranges from greater than about 1:1 to about 3:1; and
2) providing instructions for applying the composition to hair.

In certain embodiments, the cosmetic composition in the above-described method for cosmetically treating hair is for improving the manageability of hair, or for reducing/controlling the frizziness of hair or for achieving curl retention or improved curl definition in hair.

The methods according to various exemplary embodiments of the present invention may also provide improved and/or increased ease of styling or shaping hair.

It should be understood that the precise numerical values used in the specification, including the examples and claims, form additional embodiments of the invention, and are intended to include any ranges which can be narrowed to any to end points disclosed within the exemplary ranges and values provided. Efforts have been made to ensure the accuracy of the numerical values disclosed. However, any measured value can inherently contain certain errors resulting from the standard deviation found in its respective measuring technique.

### Bicarbonate Compound

The at least one bicarbonate compound of the present invention may be chosen from alkali metal or alkaline-earth metal bicarbonate compounds, The bicarbonate compound is chosen for its reactivity with regard to the associated at least one acid of the present invention. Its choice clearly falls within the competence of a person skilled in the art.

Among the examples of bicarbonate compounds that may be used in the context of the present invention, mention may be made especially of sodium bicarbonate, potassium bicarbonate, magnesium bicarbonate, calcium bicarbonate, and mixtures thereof.

Sodium bicarbonate is most particularly suitable for use in the invention.

The amount of the at least one bicarbonate compound of the present invention can be adjusted with regard to that of the associated at least one acid of the present invention. It may especially range from about 0.25% to about 9% by weight, preferably from about 0.5% to about 7% by weight, more preferably from about 1% to about 5% by weight, including all ranges and subranges therebetween.

In some embodiments, the amount of the at least one bicarbonate compound of the present invention is at about 0.5% by weight, or at about 1% by weight, or at about 2% by weight, or at about 3% by weight, or at about 4% by weight, or at about 5% by weight, relative to the total weight of the composition.

### Acid

The at least one acid compound of the present invention may be chosen from mineral or organic acids such as hydrochloric acid, orthophosphoric acid, sulfuric acid, sulfonic acids, and carboxylic acids, for instance acetic acid, tartaric acid, citric acid, lactic acid, and mixtures thereof.

The at least one acid of the present invention is preferably chosen from citric acid, lactic acid, and mixtures thereof.

In certain embodiments of the present invention, the at least one acid is citric acid.

In other embodiments of the present invention, the at least one acid is lactic acid.

The amounts of the at least one acid in the compositions of the present invention may range from about 0.25% to about 3% by weight, preferably from about 0.5% to about 3.5% by weight, more preferably from about 1% to about 2.5% by weight, relative to the total weight of the composition, including all ranges and subranges therebetween.

In some embodiments, the amount of the at least one acid in the compositions of the present invention is at about 0.25% by weight, or at about 0.3% by weight, or at about 0.5% by weight, or at about 1% by weight, or at about 1.5% by weight, or at about 2% by weight, or at about 2.5% by weight, relative to the total weight of the composition.

The amounts of the at least one bicarbonate compound may be adjusted according to the amounts of the at least one acid in the compositions of the present invention in terms of weight ratios of the at least one bicarbonate compound to the at least one acid.

In certain embodiments, the weight ratio of the at least one bicarbonate compound to the at least one acid is greater than or equal to about 1:1.

In other embodiments, the weight ratio of the at least one bicarbonate compound to the at least one acid is from about 1:1 to about 3:1, or such as from about 1.5:1 to about 3:1, or such as from about 2:1 to about 3:1, including all ranges and subranges there between.

In some embodiments, the weight ratio of the at least one bicarbonate compound to the at least one acid is at about 1:1, or is greater than about 1:1, such as at about 1.5:1, or at about 2:1, or at about 3:1.

In certain preferred embodiments, the weight ratio of the at least one bicarbonate compound to the at least one acid is about 2:1.

### Cosmetically Acceptable Carrier

The at least one cosmetically acceptable carrier of the present invention is chosen from water, organic solvents, and mixtures thereof.

The cosmetically acceptable carrier may be composed solely of water, or it may be composed of a mixture of water and at least one organic solvent.

As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, monophenyl ethers of ethylene glycol (phenoxyethanol), propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol.

Other suitable examples of organic solvents are ethylene glycol, propylene glycol, butylene glycol, hexylene glycol, propane diol, glycerin, and mixtures thereof.

The organic solvents for use in the present invention can be volatile or non-volatile compounds.

The cosmetically acceptable carrier of the present invention is typically present in an amount of from about 10% to about 98%, from about 15% to about 95%, or from about 20% to about 90% by weight, based on the total weight of the composition of the present invention, including all ranges and subranges therebetween.

pH

In some embodiments, the pH of the compositions employed in the methods of the present invention ranges from about 5 to about 10, or preferably from about 5.5 to about 9, or more preferably from about 6 to about 8.5, including all ranges and subranges therebetween.

In other embodiments, when the composition according to the present invention is in the form of a gel, the pH of the composition of the present invention is from about 6 to about 8.5, including all ranges and subranges therebetween, such as from about 6 to about 8.2.

In yet other embodiments, when the composition according to the present invention is in the form of a mousse, the pH of the composition of the present invention is about 8.

In certain embodiments, the pH of the compositions employed in the methods of the present invention is at about 8.2.

The pH of the composition of the present invention may be adjusted to the desired value using the at least one bicarbonate compound and/or the neutralizing agents of the present invention and/or other conventional acidifying or basifying agents.

All numbers expressing pH values are to be understood as being modified in all instances by the term “about” which encompasses up to +/−3%.

### Auxiliary Ingredients

The compositions of the present invention can also comprise auxiliary ingredients, for instance those chosen from the non-exhaustive list such as propellants, emulsifiers, rheology modifiers and film forming agents such as film forming polymers, neutralizing agents, humectants, conditioning agents, plasticizers, coalescers, fillers, dyes such as oxidative dyes and direct dyes, waxes, surfactants, preserving agents, oils such as mineral, organic or plant oils, fragrances, antioxidants, sunscreens, sequestering agents, softeners, antifoams, basifying agents, wetting agents, spreading agents, dispersants, pigments, proteins, ceramides, vitamins, clays, colloidal minerals, nacreous agents, peptizers, preserving agents, reducing agents, oxidizing agents, pH adjusters, silicones, plant extracts, paraffins, fatty acids, and mixtures thereof.

The person skilled in the art will ensure that any auxiliary ingredient and their amounts are selected in such a way as to cause no detriment to the properties of the compositions disclosed herein.

The at least one auxiliary ingredient may be present in an amount ranging from 0.001% to 50% by weight, relative to the total weight of the entire composition, including all ranges and subranges therebetween.

In some embodiments, the compositions of the present invention may contain at least one film forming polymer chosen from all the anionic, cationic, amphoteric and nonionic film forming polymers and mixtures thereof

In certain embodiments, the compositions of the present invention may contain at least one emulsifier.

Emulsifiers or dispersing agents, include, without limitation, any which are compatible with the solvent and ingredients used in the composition of the present invention. The emulsifying agents which can be used according to the invention are those having an HLB of less than 7 and in particular fatty acid esters of polyols such as mono-, di-, tri- or sesqui-oleates or -stearates of sorbitol or glycerol, laurates of glycerol or polethylene glycol; alkyl or alkoxy dimethicone copolyols having an alkyl or alkoxy chain pendent or at the end of a silicone-based backbone having for example from 6 to 22 carbon atoms. The emulsifying agents may also be those having an HLB greater than 7 such as fatty acid esters of polyethylene glycol (monostearate or monolaurate of polyethylene glycol); esters of fatty acids (stearate, oleate) of sorbitol which are polyoxyethylenated; polyoxy ethylenated alkyl (lauryl, cetyl, stearyl, octyl)ethers and dimethicone copolyols. In general, it is possible to use nonionic or anionic or cationic emulsifiers well known to persons skilled in the art.

The nonionic type emulsifiers are fatty acids or amides of polyalkoxylated and/or polyglycerolated fatty acids; polyoxyethylenated and/or polyoxypropylenated fatty alcohols (i.e., compounds prepared by reacting an aliphatic fatty alcohol such as behenyl or cetyl alcohol with ethylene oxide or propylene oxide or an ethylene oxide/propylene oxide mixture); fatty acid esters of polyols, optionally polyoxyethylenated and/or polyoxypropylenated (i.e., compounds prepared by reacting a fatty acid such as stearic acid or oleic acid with a polyol such as, for example, an alkylene glycol or glycerol or a polyglycerol, optionally in the presence of ethylene oxide or propylene oxide or an ethylene oxide/propylene oxide mixture); and polyalkoxylated and/or polyglycerolated alkylphenols; or polyalkoxylated and/or polyglycerolated 1,2- or 1,3-alkanediols; and alkylethers of polyalkoxylated and/or polyglycerolated 1,2- or 1,3-alkanediols or alkenediols, or mixtures thereof.

The esters of fatty acids and polyoxyethylenated polyols for which the polyol is sorbitol are known products (Polysorbate and products sold under the mark “Tween”).

The emulsifiers according to the invention can also be anionic surfactants which may have a hydrophilic-lipophilic balance (HLB) ranging from 10 to 40. They are principally salts of fatty acids (for example alkaline salts or organic salts such as amine salts), the said fatty acids having, for example, from 12 to 18 carbon atoms and being able to have a double bond as in the case of oleic acid; the alkaline salts or salts of organic bases of alkyl-sulfuric and alkyl-sulfonic acids having 12 to 18 carbon atoms, of alkyl-arylsulfonic acids whose alkyl chain contains 6 to 16 carbon atoms, the aryl group being, for example, a phenyl group. They are also ether-sulfates, in particular, the sulfatation products of fatty alcohols and polyalkoxylated alkylphenols, in which the aliphatic chain has from 6 to 20 carbon atoms and the polyalkoxylated chain has from 1 to 30 oxyalkylene units, in particular oxyethylene, oxypropylene or oxybutylene. All these anionic surfactants are well known and many among them are commercial products.

The emulsifiers according to the invention can also be cationic surfactants such as quaternary ammonium derivatives.

Particularly preferred emulsifying agents are Isoceteth-20, Polysorbate 20, PEG-40 hydrogenated castor oil, oleth-2, laureth-7, cetyl alcohol, glyceryl stearate, and mixtures thereof.

The emulsifiers may be present in the composition of the present invention in an amount of from 0.05% to 10% by weight, preferably in an amount of from 0.1 percent to 5% by weight, and more preferably in an amount of from 0.5% to 1.0% by weight, based on the total weight of the composition.

The emulsifiers may be employed in the compositions of the present invention in order to solubilize fatty substances such as fragrance oils or esters, whenever said fatty substances are additionally present in the compositions.

In other embodiments, the compositions of the present invention may contain at least one rheology modifier (also called rheology-modifying agent).

Broadly, the rheology modifier(s) that may be useful in the practice of the present invention include those conventionally used in cosmetics such as polymers of natural origin and synthetic polymers. Rheology modifiers are employed in the compositions of the present invention when it is desired to adjust the viscosity or thickness of the compositions or to achieve a particular composition texture.

Representative rheology-modifying agents that may be used in the practice of the present invention are those other than the at least one film forming polymer of the present invention and include nonionic, anionic, cationic, and amphoteric polymers, and other rheology modifiers such as cellulose-based thickeners (e.g., hydroxyethylcellulose, hydroxypropylcellulose, carboxymethylcellulose, cationic cellulose ether derivatives, quaternized cellulose derivatives, etc.), guar gum and its derivatives (e.g., hydroxypropyl guar, cationic guar derivatives, etc.), gums such as gums of microbial origin (e.g., xanthan gum, scleroglucan gum, etc.), and gums derived from plant exudates (e.g., gum arabic, ghatti gum, karaya gum, gum tragacanth, carrageenan gum, agar gum and carob gum), pectins, alginates, and starches, crosslinked homopolymers of acrylic acid or of acrylamidopropane-sulfonic acid, associative polymers, non-associative thickening polymers, and water-soluble thickening polymers.

In some embodiments, the rheology-modifying agent includes a polymer other than the at least one film forming polymer of the present invention and chosen from nonionic, anionic, cationic and amphoteric amphiphilic polymers.

The rheology-modifying agents may also be chosen from associative celluloses include quaternized cationic celluloses and quaternized cationic hydroxyethylcelluloses modified by groups containing at least one hydrophobic chain, such as alkyl, arylalkyl or alkylaryl groups containing at least 8 carbon atoms, and mixtures thereof.

The alkyl radicals carried by the above quaternized celluloses or hydroxyethylcelluloses may, in various embodiments, comprise from 8 to 30 carbon atoms. The aryl radicals may, for example, denote the phenyl, benzyl, naphthyl or anthryl groups. Representative examples of quaternized alkylhydroxy-ethylcelluloses containing a C8-C30 hydrophobic chain include the products Quatrisoft LM 200®, Quatrisoft LM-X 529-18-A®, Quatrisoft LM-X 529-18B® (C12 alkyl) and Quatrisoft LM-X 529-8® (Ci8 alkyl) sold by Amerchol and the products Crodacel QM®, Crodacel QL® (C12 alkyl) and Crodacel QS® (Ci8 alkyl) sold by Croda.

Representative examples of nonionic cellulose derivatives include hydroxyethylcelluloses modified by groups comprising at least one hydrophobic chain, such as alkyl, arylalkyl or alkylaryl groups, or their blends, and in which the alkyl groups are, for example, C8-C22 alkyl groups, such as the product Natrosol Plus Grade 330 CS® (C16 alkyls) sold by Aqualon or the product Bermocoll EHM 100® sold by Berol Nobel.

Representative examples of cellulose derivatives modified by alkylphenyl polyalkylene glycol ether groups include the product Amercell Polymer HM-1500® sold by Amerchol.

The rheology-modifying agent is typically present in an amount ranging from about 0.01% to about 10% by weight, in some embodiments from about 0.1% to about 5% by weight, or from about 0.5% to about 1% by weight, based on the total weight of the composition.

In some instances, certain rheology modifiers are also known as gelling agents or thickening agents.

In yet other embodiments, the compositions of the present invention may contain at least one propellant. Propellants can used to deliver the composition as a foam (such as in a mousse product).

Representative examples of propellants include C3 to C5 alkanes such as n-butane, isobutane, isopropane, and propane, dimethyl ether, C2-C5 halogenated hydrocarbons, e.g., 1,1-difluoroethane or hydroflurocarbon, difluoroethane, chlorodifluoroethane, chlorodifluoromethane, air (such as compressed air), nitrogen, carbon dioxide, and mixtures thereof. The amount of the propellant can range from about 3 to about 90%, and in some embodiments from about 3 to about 60%, by weight, or such as from about 3 to about 20% by weight, or such as from about 3 to about 10% by weight, or such as from about 3 to about 6%, by weight based on the total weight of the composition, including all ranges and subranges therebetween.

The compositions in the methods of the present invention may take the form of a gel, a mousse such as an aerosol mousse, a spray such as an aerosol spray or a pump spray, a spray gel, a lotion, a tonic, or a cream. The compositions may also be provided as rinse-off or leave-in products, preferably, leave-in products.

In one preferred embodiment, the composition of the present invention is used as a leave-in product.

In another preferred embodiment, the composition of the present invention is used as a hair styling product.

In one particular embodiment, the composition of the present invention is in the form of a gel.

In another particular embodiment, the composition of the present invention additionally contains at least one propellant and is in the form of a mousse.

In some embodiments, the composition of the present invention is a hair cosmetic product such as hair styling product or an anti-frizz product or hair smoothing/straightening product or a product that provides/improves curl definition or curl retention.

In some other embodiments, the cosmetically acceptable carrier in the composition of the present invention comprises at least one volatile organic solvent or compound (VOC) (e.g., in the case of a spray or an aerosol spray). To reduce the amount of VOC (low VOC product), the volatile organic solvent or compound is partially replaced with water. The amount of the volatile organic solvent generally ranges from greater than 0 (e.g., about 0.01%) to about 90%, and in some embodiments from greater than 0 to about 55%, and in some embodiments from greater than 0 to about 2%, by weight, based on the total weight of the composition. It is preferred that the amount of volatile organic solvent does not exceed 55% by weight.

The compositions of the present invention may be packaged, for example, in a bottle, a spray device such as an aerosol container/can, a pump dispenser or pump spray, a jar, such as those customary in cosmetology.

The compositions may be applied onto keratin fibers by using the fingers or hand, or by use of a suitable applicator or by directly dispensing the compositions from a device.

### Methods of Making

The compositions of the present invention are made by combining at least one bicarbonate compound, at least one acid, at least one cosmetically acceptable carrier, and optionally, at least one auxiliary ingredient, wherein the weight ratio of the at least one bicarbonate compound (a) to the at least one acid (b) is greater than or equal to about 1:1.

In one embodiment, the method of making the cosmetic composition of the present invention comprises the steps of:

a) mixing at least one acid (e.g., citric acid, lactic acid or mixtures thereof) with at least one cosmetically acceptable carrier (e.g., water or a water/organic solvent mixture) to form an acid-carrier mixture; and

b) adding at least one bicarbonate compound (e.g., sodium bicarbonate) to the acid-carrier mixture while mixing is continued; and

wherein the weight ratio of the at least one bicarbonate compound (a) to the at least one acid (b) is greater than or equal to about 1:1.

The at least one bicarbonate compound in any one of the above-described methods of making may be employed in an amount of from about 0.25% to about 9% by weight, such as from about 1% to about 2.5% by weight, relative to the total weight of the composition, including all ranges and subranges therebetween.

The at least one acid in any one of the above-described methods of making may be employed in an amount of from about 0.25% to about 3% by weight, such as from about 1% to about 2.5% by weight, relative to the total weight of the composition, including all ranges and subranges therebetween.

### Methods of Use

An embodiment disclosed herein is a method for cosmetically treating hair, or for improving the manageability of hair or controlling/reducing the frizziness of hair or improving the curl definition or curl retention in hair, involving applying onto hair, any one of the compositions disclosed herein.

The compositions of the present invention may be employed in an effective amount to adequately cover the surface of the fibers of the hair and to achieve the desired cosmetic effect such as hair manageability, frizz control, volume reduction, and curl retention. The precise amount of composition to be applied onto the hair will thus depend on the degree of treatment desired.

Thus, in one embodiment, the present invention relates to a method for improving the manageability of hair, the method comprising:

1) providing a cosmetic composition, containing:

a) from about 1% to about 5% by weight of sodium bicarbonate;

b) from about 1% to about 2.5% by weight at least one acid selected from citric acid, lactic acid, and mixtures thereof;

c) a cosmetically acceptable carrier selected from water and water/organic solvent mixture; and

d) optionally, at least one auxiliary ingredient selected from at least one emulsifier, at least one rheology modifier; at least one propellant, at least one film forming agent, and mixtures thereof;

wherein the weight ratio of the at least one bicarbonate (a) to the at least one acid (b) ranges from greater than about 1:1 to about 2:1; and wherein all weights are relative to the total weight of the composition; and
2) providing instructions for applying the composition to hair.

In another embodiment, the present invention relates to a method for improving the manageability of hair, the method comprising applying onto hair, a cosmetic composition, containing:

a) from about 1% to about 5% by weight of sodium bicarbonate;

b) from about 1% to about 2.5% by weight at least one acid selected from citric acid, lactic acid, and mixtures thereof;

c) a cosmetically acceptable carrier selected from water and water/organic solvent mixture; and

d) optionally, at least one auxiliary ingredient selected from at least one emulsifier, at least one rheology modifier; at least one propellant, at least one film forming agent, and mixtures thereof;

wherein the weight ratio of the at least one bicarbonate (a) to the at least one acid (b) ranges from greater than about 1:1 to about 3:1.

The hair that has been contacted with the compositions of the present invention may be heated and/or styled or shaped using a blow dryer, flat iron, heating implement, or other suitable devices, and/or by combing or brushing or running the fingers through the hair.

Instructions for applying the compositions disclosed herein may comprise directions of use of the composition for the end-user to follow. The end-user may be a consumer or cosmetologist or salon hair dresser. Directions may comprise instructing the end-user to take an amount of the composition in sufficient quantity such that the composition adequately covers the hair fibers and imparts the desired shape or style or hold to the hair fibers. Directions may additionally instruct the end-user to use a device such as a comb, brush (e.g., hair brush or brush wand), flat iron plates or the fingers for shaping or styling the hair or for separating the fibers of the hair. Directions may also additionally instruct the end-user to apply heat to the hair such as by blow drying the hair or using a heating device on the hair.

It has been surprisingly and unexpectedly discovered that the methods of the present invention resulted in satisfactory hair manageability, frizz control and curl retention.

The degree of hair manageability, frizz control and curl retention on the hair may be evaluated by assessing the appearance of the hair, or the reduction in frizziness or volume of the hair, or the retention or curl of the hair after contacting the hair with the composition of the invention.

As used herein, the method and composition disclosed herein may be used on the hair that has not been artificially dyed, pigmented or permed or on the hair that has been artificially dyed, pigmented or permed.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result.

### EXAMPLES

The following Examples are intended to be non-restrictive and explanatory only, with the scope of the invention being defined by the claims.

The ingredient amounts/concentrations in the compositions/formulas described below are expressed in % by weight, based on the total weight of the composition/formula.

EXAMPLE 1: Formula A

| INCI US Name | Concentration |
|---|---|
| WATER | 92.50 |
| CITRIC ACID | 2.50 |
| SODIUM BICARBONATE | 5.00 |

The formula above was made according to this method of preparation:

1. Water was added to a beaker or container and agitation/mixing was commenced.

2. Acid was added to the water and mixed until dissolved.

3. Sodium bicarbonate was added while continuing to mix.

4. The mixture began to foam and was mixed until the foam dissipated.

5. If desired, auxiliary ingredients and additives can be added.

The formula A above was tested on hair swatches (curly hair; all swatches have the same degree of curl and length before the test).

Test I:

Three curly hair swatches were tested as follows:

The swatches were cleansed with shampoo. 3 grams of the formula A were applied on each of two wet swatches (invention). The third swatch was treated with de-ionized water (control swatch). All swatches were left to dry overnight. Once dry, all three swatches were placed in humidity chamber at 80% relative humidity for 8 hours. The two swatches treated with the formula A showed enhanced curl definition with very little frizz. The control swatch treated with de-ionized water had very little curl definition and a lot of frizz was observed. The overall diameter (area coverage) of the control swatch was much larger than the diameter of the other two swatches treated with Formula A, i.e., the hair treated with Formula A showed less volume.

Test II:

Five curly hair swatches were tested as follows:

The swatches were cleansed with shampoo. 3 grams of the formula A were applied on each of two wet swatches (invention). 3 grams of a composition containing 5% sodium bicarbonate were applied on each of two other wet swatches (comparative). The fifth swatch was treated with de-ionized water (control swatch). All swatches were left to dry overnight. Once dry, all three swatches were placed in humidity chamber at 80% relative humidity for 8 hours.

The control swatch treated with de-ionized water exhibited very little curl definition and a lot of frizz than the other test swatches. It also exhibited more volume compared to the other test swatches as evidenced by the much larger overall diameter (area coverage) of the control swatch compared to the diameter of the swatches treated with formula A and with the comparative.

In addition, the two swatches treated with formula A showed enhanced curl definition with very little frizz and less volume compared to the swatches treated with the comparative. The swatches treated with formula A and the comparative exhibited more elongation or straightening compared to the control swatch. Although the swatches treated with the comparative exhibited more elongation or straightening compared to the swatches treated with Formula A, the swatches treated with the comparative were more frizzy and had more volume and less curl definition compared to those treated with Formula A.

It will be apparent to those skilled in the art that various modifications and variations can be made in the delivery system, composition and methods of the invention without departing from the spirit or scope of the invention. Thus, it is intended that the present invention cover the modifications and variations of this invention provided that they come within the scope of the appended claims and their equivalents.

What is claimed is:

1. A method for improving the manageability of hair, the method comprising:
1) providing a cosmetic composition, containing:
a) from about 0.25% to about 9% by weight of at least one bicarbonate compound
b) from about 0.25% to about 3% by weight at least one acid;
c) a cosmetically acceptable carrier selected from water and water/organic solvent mixture; and
d) optionally, at least one auxiliary ingredient selected from at least one emulsifier, at least one rheology modifier; at least one propellant, at least one film forming agent, and mixtures thereof;
wherein the weight ratio of the at least one bicarbonate (a) to the at least one acid (b) ranges from greater than or equal to about 1:1; and
wherein all weights are relative to the total weight of the composition; and
2) providing instructions for applying the composition to hair.

2. The method of claim 1, wherein the at least one bicarbonate compound (a) is present in an amount of from about 0.25% to about 9% by weight, relative to the total weight of the composition.

3. The method of claim 2, wherein the at least one acid (b) is present in an amount of from about 0.25% to about 3%, by weight relative to the total weight of the composition.

4. The method of claim 3, wherein the at least one cosmetically acceptable carrier (d) is present in an amount of from about 10% to about 98% by weight, relative to the total weight of the composition.

5. The method of claim 4, wherein the cosmetically acceptable carrier is selected from water, organic solvents, and mixtures thereof.

6. The method of claim 1, wherein the weight ratio of the at least one bicarbonate compound (a) to the at least one acid (b) is greater than about 1:1

7. The method of claim 6, wherein the weight ratio of the at least one bicarbonate compound (a) to the at least one acid (b) is from about 2:1 to about 3:1.

8. The method of claim 7, wherein the weight ratio of the at least one bicarbonate compound (a) to the at least one acid (b) is about 2:1.

9. The method of claim 6, wherein the at least one bicarbonate (a) is sodium bicarbonate.

10. The method of claim 6, wherein the at least one acid (b) is selected from citric acid, lactic acid, and mixtures thereof.

11. The method of claim 10, wherein the composition further comprises at least one auxiliary ingredient selected from propellants, emulsifiers, rheology modifiers and film forming agents, neutralizing agents, humectants, conditioning agents, plasticizers, coalescers, fillers, dyes such as oxidative dyes and direct dyes, waxes, surfactants, preserving agents, oils such as mineral, organic or plant oils, fragrances, antioxidants, sunscreens, sequestering agents, softeners, antifoams, basifying agents, wetting agents, spreading agents, dispersants, pigments, proteins, ceramides, vitamins, clays, colloidal minerals, nacreous agents, peptizers, preserving agents, reducing agents, oxidizing agents, pH adjusters, silicones, plant extracts, paraffins, fatty acids, and mixtures thereof.

12. The method of claim 1, wherein the method is for controlling or reducing the frizziness of hair.

13. The method of claim 1, wherein the method is for improving the curl retention of hair.

14. The method of claim 1, wherein the composition is a hair cosmetic composition for styling hair or for controlling or reducing the frizziness of hair or for retaining the curl of hair.

15. A method for improving the manageability of hair, the method comprising applying onto hair, a cosmetic composition, comprising:
a) from about 1% to about 5% by weight of sodium bicarbonate;
b) from about 1% to about 2.5% by weight at least one acid selected from citric acid, lactic acid, and mixtures thereof;
c) a cosmetically acceptable carrier selected from water and water/organic solvent mixture; and
d) optionally, at least one auxiliary ingredient selected from at least one emulsifier, at least one rheology modifier; at least one propellant, at least one film forming agent, and mixtures thereof;
wherein the weight ratio of the at least one bicarbonate (a) to the at least one acid (b) ranges from greater than about 1:1 to about 3:1.
